(19) United States
(12) Patent Application Publication
(10) Pub. No.: US 2018/0318027 A1
(43) Pub. Date: Nov. 8, 2018

(54) DISPOSABLE SURGICAL DRAPE

(71) Applicant: ART, LIMITED, Grand Cayman (KY)

(72) Inventor: Takayuki Akahoshi, Tokyo (JP)

(21) Appl. No.: 15/970,957

(22) Filed: May 4, 2018

Related U.S. Application Data
(60) Provisional application No. 62/510,888, filed on May 25, 2017.
(60) Provisional application No. 62/501,187, filed on May 4, 2017.

(51) Int. Cl. A61B 46/20; A61B 46/00

(52) U.S. Cl. CPC A61B 46/20; A61B 2046/205; A61B 46/40

(57) ABSTRACT

A disposable surgical drape is provided for use while performing surgical procedures on an eye of a patient, or other surgical procedures. There is an operative, surgical aperture formed through a base sheet for being located proximal to a first eye, or other surgical site, of the patient. An attachment zone is formed on the base sheet, proximal to the surgical aperture, and at least partially surrounds the surgical aperture. In one form, a guide extends across the surgical aperture on the upper surface of the base sheet. In another form, the base sheet has a non-surgical aperture spaced from the surgical aperture for being located proximal to a second eye of the patient. The disposable surgical drape can be provided in a folded form to facilitate placement on a patient and subsequent unfolding and spreading of the drape. In this embodiment, the drape includes a minimally-adhesive film that defines the surgical aperture.

### CROSS-REFERENCES TO RELATED APPLICATIONS

This application claims priority of U.S. provisional patent application Ser. No. 62/510,888, filed May 25, 2017, and U.S. provisional patent application Ser. No. 62/501,187, filed May 4, 2017, and those prior patent applications are incorporated here by reference in their entirety to provide continuity of disclosure, and applicant claims the benefit of those prior applications.

### FIELD OF THE INVENTION

The present invention relates generally to the field of surgery including ophthalmic surgery, and more particularly, to a disposable drape for use during surgery of the human eye, as well for use during other surgical procedures. In one aspect of the invention, the surgical drape is provided an a specifically folded configuration that facilitates initial placement of the drape on a patient and subsequent spreading of the drape to cover the patient. In another aspect, the drape includes a minimally-adhesive film, which may be pre-cut, to provide an attachment zone for the drape.

### BACKGROUND OF THE INVENTION

When the eye becomes aged, diseased, or injured it may be necessary to remove the natural lens of the eye. Such removal is common for cataract surgery, in which a lens that has become clouded is removed. The removal of the natural lens of the eye may result in the loss or alteration of focused vision of a patient. Eyeglasses, contact lenses, or the implantation of an artificial lens may be necessary to restore the vision of the patient.

During the removal of the natural lens of the eye, a physician will typically make one or more small incisions in the eye and insert a vibratory needle into the incision. The needle may be vibrated at ultrasonic speeds in torsional, longitudinal, elliptical, or blended modes in order to dismember the lens in a process known as phacoemulsification. The needle typically contains one or more aspiration passages for removing the broken lens particles from the eye. Lasers may also be used to dismember the lens.

This surgical process is typically performed with an ophthalmic drape being placed over the eye of the patient. The ophthalmic drape has one aperture for being placed over the eye upon which surgery will occur. The drape has an aperture for insertion of the surgical equipment, and may have an adhesive to secure the drape to the patient's skin. A topical or local anesthetic is applied to the eye. However, general anesthesia may be required for some patients who cannot remain still for the surgical procedure or for some patients who may suffer from claustrophobia. The obstructed vision of the eye which is not being operated upon may further exacerbate such claustrophobia. General anesthetic can add substantial expense to the surgical operation and may further present risks to the health of the patient, when compared to local anesthetic.

### SUMMARY OF THE INVENTION

The present invention is directed to a disposable surgical drape with a wide range of potential applications. The inventor of the present invention has discovered the present drape can made particularly suitable for ophthalmic surgical procedures, and can be configured for minimizing the claustrophobia of a patient undergoing ophthalmic surgery. In another aspect of the invention, the surgical drape is provided in a folded configuration to facilitate placement of the drape on a patient, and subsequent spreading of the drape to cover the patient. In another aspect of the invention, the drape includes a minimally-adhesive film, which may be pre-cut, to provide an attachment zone for facilitating fitment of the drape at the patient's eye. The provision of the minimally-adhesive film permits use of the drape for other surgical procedures.

In accordance with the present invention, a disposable surgical drape is provided, which is particularly useful for ophthalmic surgery. The drape has a base sheet of thin, flexible material. The base sheet has an upper surface, a lower surface for contacting a patient, and at least one edge. The drape has a surgical aperture formed through the base sheet for being located proximal a first eye of the patient. The drape further has an attachment zone on the base sheet lower surface, which is proximal the surgical aperture. The attachment zone at least partially surrounds the surgical aperture for attaching the base sheet lower surface to the patient. The drape may further include a non-surgical aperture formed through the base sheet. The non-surgical aperture is for being located proximal a second eye of the patient.

Another feature of the present invention is to provide a low-cost, disposable surgical drape for the reduction and/or minimization of claustrophobia in a patient.

In another aspect of the present invention, a folded surgical drape is provided to facilitate positioning of the drape on a patient, and subsequent spreading of the drape of over the patient.

In accordance with this aspect of the invention, the folded surgical drape comprises a rectangular base sheet of thin, flexible material, preferably a non-woven fabric, wherein the base sheet includes an upper surface, and a lower surface for contacting the patient. The rectangular sheet includes a least one edge, typically opposite upper and lower edges, and opposite side edges.

The drape includes an attachment zone on the lower surface of the base sheet which is positionable at a surgical site of a patient. The attachment zone preferable comprises a minimally adhesive film for attaching the lower surface of the base sheet to the patient.

The attachment zone of the drape can be provided with a surgical aperture, which can optionally be pre-cut, or formed in the drape at the time it is placed on the patient. The attachment zone surrounds the surgical aperture, which facilitates access to the surgical site of the patient.

The attachment zone of the drape is positioned intermediate of a first pair of opposite, marginal portions of the drape, and intermediate a second pair of opposite, marginal portions of said drape, with the attachment zone thus positioned generally centrally of the drape.

In accordance with this aspect of the invention, each of the marginal portions of the drape includes at least one fold, to thereby facilitate placement of the folded drape on a patient with the attachment zone positioned at the surgical site of the patient. Thereafter, the first and second pairs of marginal portions can be unfolded to spread said drape to cover the patient.

In the preferred embodiment, each of the marginal portions comprises a plurality of folds, with it being particularly preferred that each plurality of folds comprises accordion-folds, wherein the drape material is folded back and forth on itself to form a plurality of folded pleats. Notably, the accordion-folds in the first pair of marginal portions are formed prior to the accordion-folds in the second pair of marginal portions, so that the accordion-folds in the second pair of marginal portions overlap, and further fold, the accordion-folds in first pair marginal portions. It is particularly preferred that the first pair of marginal portions be respectively positioned above and below the surgical aperture in the drape, with the second pair of marginal portions being unfolded first to spread the drape laterally over the patient laterally, and the first pair of marginal portions thereafter being unfolded to spread the drape over the patient above and below the first eye.

In another aspect of the present invention, a non-surgical aperture is formed through the base sheet, the non-surgical aperture for being located proximal a second eye of the patient. As noted, the provision of second aperture can desirably act to minimize claustrophobic sensations experienced by the patient.

A method of folding an ophthalmic drape in accordance with the present invention is also disclosed.

Other features and advantages of the present invention will be better understood with reference to the accompanying figures and detailed description.

### BRIEF DESCRIPTION OF THE DRAWINGS

FIG. 1 is a bottom plan view of a prior art ophthalmic drape;

FIG. 2 is a top plan view of a first embodiment of a disposable surgical drape according to the present invention;

FIG. 3 is a section view along 3-3 in FIG. 2;

FIG. 4 is a section view along 4-4 in FIG. 2;

FIG. 5 is a bottom plan view of the surgical drape of FIG. 2;

FIG. 6 is a bottom plan view of a second embodiment of a surgical drape according to the present invention;

FIG. 7 is a bottom plan view of a third embodiment of a surgical drape according to the present invention;

FIG. 8 is a top plan view of a fourth embodiment of a surgical drape according to the present invention.

FIG. 9 is a top plan view of a fifth embodiment of a surgical drape according to the present invention;

FIG. 10 is a partial, enlarged top plan view of a variation of the surgical drape shown in FIG. 9;

FIG. 11 is a top plan view of a sixth embodiment of a surgical drape according to the present invention;

FIG. 12 is a partial, enlarged top plan view of a variation of the surgical drape shown in FIG. 11;

FIG. 13 is a top plan view of the present surgical drape prior to folding of the drape to a compacted form for positioning on a patient;

FIG. 14 is a relatively enlarged, fragmentary view of the surgical drape shown in FIG. 13 showing a surgical aperture of the drape;

FIG. 15 is a diagrammatic, sequential view show steps of folding the surgical drape shown in FIG. 13;

FIG. 16 is a diagrammatic view showing a partially folded condition of this embodiment of the present surgical drape;

FIG. 17 is a diagrammatic view showing the present surgical drape in a fully folded condition, ready for positioning on a patient for subsequent spreading and draping on the patient; and

FIGS. 18-20 are a series of illustrations showing the use of the present surgical drape, including the embodiment with a pre-cut minimally-adhesive film.

### DETAILED DESCRIPTION OF THE PREFERRED EMBODIMENTS

While the present invention is susceptible of embodiment in various forms, there is shown in the drawings and will hereinafter be described presently preferred embodiments, with the understanding that the present disclosure is to be considered an exemplification of the invention, and is not intended to limit the invention to the specific embodiments illustrated.

The present disposable surgical drape is particularly suited for use in connection with ophthalmic surgical procedures. However, features of the surgical drape permit it be advantageously used in other procedures, including central venous catheterization (CVC), other catheterization procedures, anesthesia procedures, and other surgical procedures, as will be described.

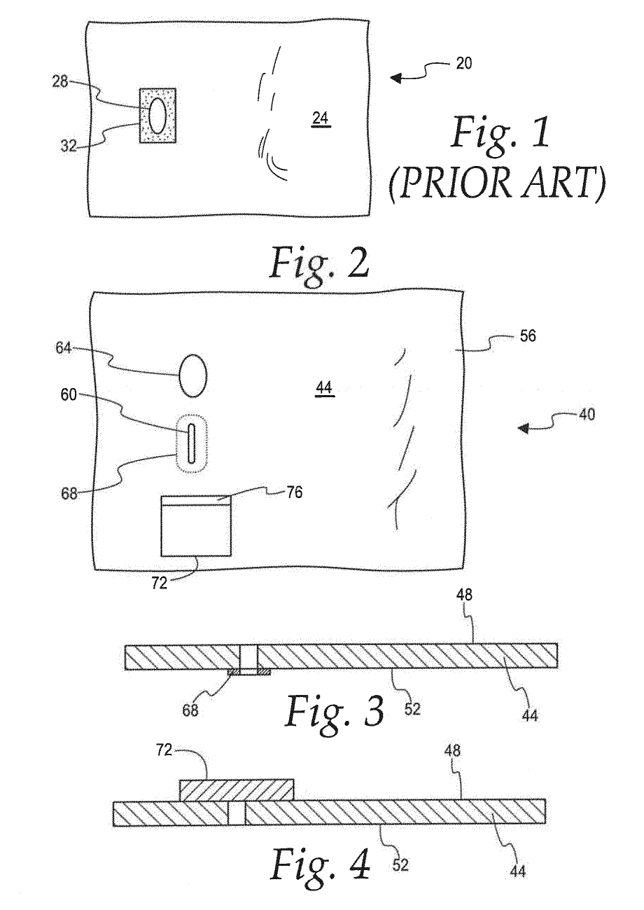

FIG. 1 shows a bottom plan view of a prior art ophthalmic drape 20. Drape 20 is formed from a generally rectangular base sheet 24 made from a thin, flexible material. Base sheet 24 can be made from tissue paper, textiles, fluid-permeable and non-fluid permeable polymers, and/or composites thereof.

As seen in FIG. 1, the base sheet 24 has an aperture 28 extending through the base sheet 24 for being positioned over an eye of a patient during ophthalmic surgery. The aperture 28 is typically initially formed in the base sheet 24, cut from the base sheet 24, or is subsequently created by tearing or rupturing of the base sheet 24 along a weakened or perforated area. The aperture 28 can also be formed by incision or piercing of the base sheet 24 by a surgical instrument. The aperture 28 is typically in the shape of a circle, oval, or elongate slot shape.

As can further be seen in FIG. 1, the drape 20 further has an adhesive 32 surrounding the aperture 28 so that the base sheet 24 can be affixed to the face of the patient.

FIG. 2 is a top plan view of a first embodiment of a disposable surgical drape 40, which is particularly useful for ophthalmic procedures, according to the present invention. The drape 40 is formed from a generally rectangular base sheet 44 made from a thin, flexible material. Base sheet 44 can be made from tissue paper, textiles, fluid-permeable and non-fluid permeable polymers, and/or composites thereof. Preferably, the base sheet 44 is constructed from a light, water resistant paper that will be disposed after a single use. The drape 40 need not have a generally rectangular base sheet 44, and may have a variety of shapes, such as polygonal, arcuate, or irregular shapes. The base sheet 44 has an upper surface 48 (as best shown in FIG. 56, a lower surface 52 (as best shown in FIG. 3), and an edge 56 therebetween. The lower surface 52 is for being positioned upon and contacting the face of a patient undergoing an ophthalmic surgery. In another presently preferred form, the base sheet 24 comprises a composite non-woven SMS (spunbond-meltblown-spunbond) fabric.

As seen in FIG. 2, the base sheet 44 has a surgical aperture 60 extending through the base sheet 44 for being positioned over an eye of a patient, which is to be operated upon by a physician. The surgical aperture 60 may be initially formed in the base sheet 44, cut from the base sheet 44, may be subsequently created by tearing or rupturing of the base sheet 44 along a weakened or perforated area. The surgical aperture 60 may also be formed by incision or piercing of the base sheet 44 by a surgical instrument. The surgical aperture 60 has the form of an elongate slot, but may have a variety of shapes such as circular, oval, polygonal, or irregular shapes. When the surgical aperture 60 has the form of the elongate slot, both upper and lower lids can be perfectly covered by the drape quite easily and to reduce or prevent the risk of infection from the dirty eye lashes or Meibomian secretion.

As can further be seen in FIG. 2, the drape 40 further has a second or non-surgical aperture 64 extending through the base sheet 44 for being positioned over the second eye of a patient, which is not to be operated upon by a physician. The non-surgical aperture 64 may be initially formed in the base sheet 44, cut from the base sheet 44, may be subsequently created by tearing or rupturing of the base sheet 44 along a weakened or perforated area. The non-surgical aperture 64 has the form of an oval, but may have a variety of shapes such as circular, polygonal, or irregular shapes. Preferably, the non-surgical aperture 64 is sized to provide an unobstructed view to the second eye of the patient. Further, it is preferred that the non-surgical aperture 64 has a larger surface area than surgical aperture 60. Another important feature of the drape 40 is that the eye position can be better controlled by the fixation light seen by the non-operated eye. In the prior art, the conventional fixing light was incorporated into a microscope to control the operated eye position during the surgery. However, due to the bright illumination of the microscope and poor visual acuity of the operated eye, such a fixing light was not ideal. When the non-operated eye can see through the non-surgical aperture 64, the non-operated eye can be effective in leading the eye position during surgery.

As shown in FIG. 2, the surgical aperture 60 is for being located over the right eye of a patient, and the non-surgical aperture 64 is for being located over the left eye of the patient. However, it will be understood that the non-surgical aperture 64 may be located over the right eye of the patient, while the surgical aperture 60 is located over the left eye of the patient-depending on which eye is to be operated upon.

The inventor has found that providing a base sheet 44 with a non-surgical aperture 64 in addition to a surgical aperture 60 may minimize the anxiety of claustrophobic patients because the vision of the second eye is not obscured by the base sheet 44. Provision of such stereoscopic vision to the patient may provide a low-cost alternative to general anesthetic and may reduce the movement of an anxious, claustrophobic patient.

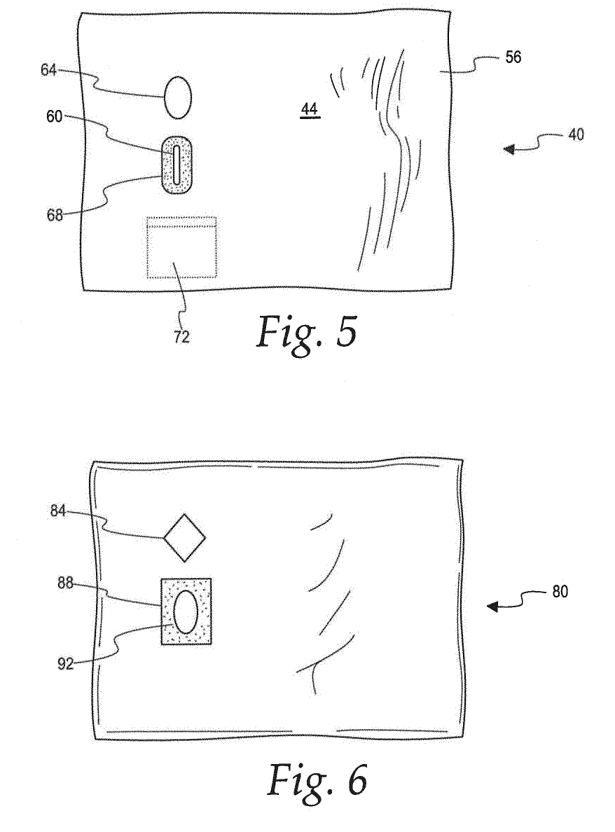

As can further be seen in FIG. 5, the drape 40 further has an attachment zone 68 located on the base sheet lower surface 52. The attachment zone 68 is located proximal the surgical aperture 60 and fully surrounds the surgical aperture 60. The attachment zone 68 is for attaching the base sheet lower surface 52 to the patient and may be formed from an adhesive applied to the base sheet 44, layer or layers of tape applied to the lower surface 52, or a hook and loop type attachment for being mated with a separate hook and loop attachment that is adhesively affixed to the face of the patient. The attachment zone 68 also serves completely cover the eye lids and the eye lashes of the patient in order to prevent infection caused by the bacteria secreted with the Meibomian gland. While the attachment zone 68 is shown fully surrounding the surgical aperture 60, the attachment zone 68 may alternatively be provided only partially surrounding surgical aperture, or may be located elsewhere on the base sheet lower surface 52, such as proximal the non-surgical aperture 64.

As can further be seen in FIG. 2, the drape 40 further has a pouch or compartment 72 on the base sheet upper surface 48. The compartment 72 is located proximal the surgical aperture 60 with an opening 76 facing toward the surgical aperture 60. The compartment 72 is designed to catch any overflow of balanced salt solution that may not be aspirated into the surgical devices used in the surgical aperture 60. Typically, the balanced salt solution will be applied to the eye undergoing surgery as an irrigation solution. The balanced salt solution is then typically aspirated through one or more lumens for sanitary disposal. Excess balanced salt solution that is not aspirated into such lumens may collect on the base sheet 44. The compartment 72 helps to eliminate or at least control the accumulation of balanced salt solution. Alternatively, the compartment 72 could retain various surgical tools, equipment, and/or supplies when the drape 40 is draped over and attached to the face of the patient. The compartment 72 may be integrally formed with the drape via heat welding, adhesive, or stitching to the base sheet upper surface 48.

FIG. 3 shows a section view of the drape 40 taken along view line 3-3. This view of the drape 40 is taken through the surgical aperture 60 and shows the attachment zone 68 in the form of a layer of tape applied to the lower surface 52 of the base sheet 44.

FIG. 4 shows another section view of the drape 40, which is taken along view line 4-4. This view of the drape 40 is taken through the compartment 72 and shows the compartment 72 in the form of a layer of a separate piece of material that is heat welded along its edges to the upper surface 48 base sheet 44.

FIG. 6 illustrates a second embodiment of an ophthalmic drape 80 according to the present invention. The second embodiment of the drape 80 functions in the same manner as the first embodiment of the drape 40 discussed above, except that the second embodiment of the drape 80 has a non-surgical aperture 84 with in the form of parallelogram. Furthermore, the drape 80 has an adhesion zone 88 in having a polygonal shape and surrounding the surgical aperture 92.

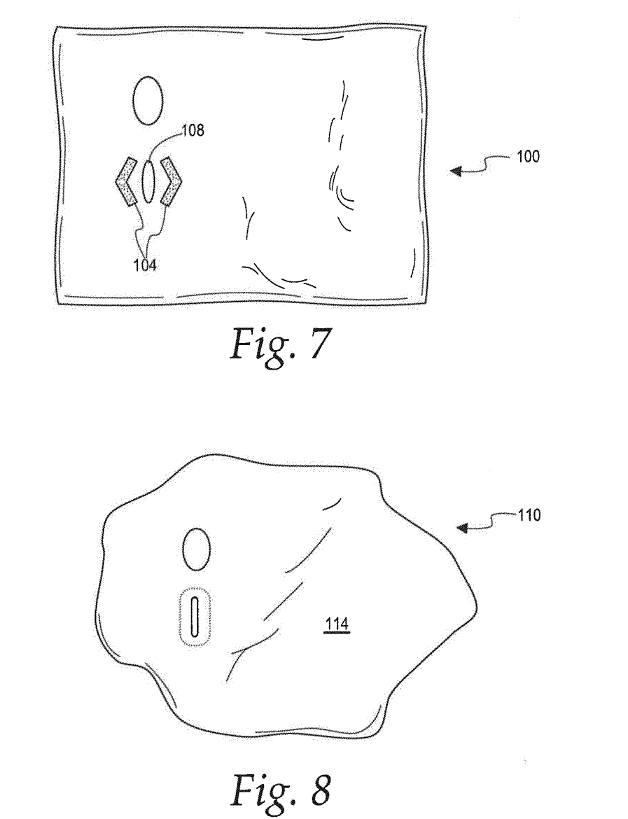

FIG. 7 illustrates a third embodiment of an ophthalmic drape 100 according to the present invention. The third embodiment of the drape 100 functions in the same manner as the first embodiment of the drape 40 discussed above, except that the third embodiment of the drape 100 has an adhesion zone 104 in the form of two distinct zones and only partially-surrounding the surgical aperture 108.

FIG. 8 illustrates a fourth embodiment of an ophthalmic drape 110 according to the present invention. The fourth embodiment of the drape 110 functions in the same manner as the first embodiment of the drape 40 discussed above, except that the fourth embodiment of the drape 110 has a base sheet 114 that has an irregular shape.

The method of use of the inventive ophthalmic drape 40 will now be discussed. Drapes 80,100, and 110 function in the same manner as described hereinafter with respect to drape 40. The drape 40 is removed from any accompanying packaging or dispenser, which forms no part of the present invention. The attachment zone 68 is activated when the adhesive tape is exposed by the user and the drape lower surface 52 is placed on the skin of the patient with the surgical aperture 60 centered on a first eye of the patient. The first eye is the eye to be operated upon. The drape 40 is placed on the patient such that the non-surgical aperture 64 is also centered on the second eye of the patient. The second eye is the eye that will not be operated upon. The user will ensure that the non-surgical aperture 64 and the surgical aperture 60 are positioned such that the patient is able to maintain normal, stereoscopic vision. If the compartment 72 is provided on the base sheet upper surface 48, then the user may place surgical equipment and/or supplies in the compartment 72 after the drape 40 is attached to the patient. The drape 40 hangs on the face of the patient such that the compartment opening 76 will face upright, away from the ground, so as to be accessible to the user of the drape 40. If the surgical aperture 60 is formed subsequent to the breach of a perforated or weakened area in the base sheet 44, then the user will breach the base sheet 44 and form the surgical aperture 60.

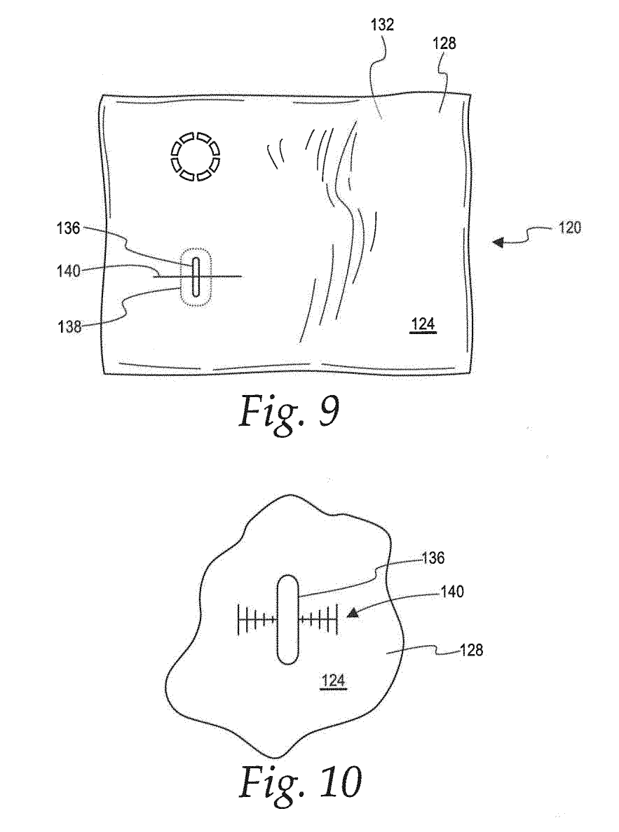

FIG. 9 illustrates a fifth embodiment of an ophthalmic drape 120 according to the present invention. The fifth embodiment of the drape 120 functions in the same manner as the first embodiment of the drape 40 discussed above, and the drape 120 has a base sheet 124 with an upper surface 128, a lower surface 132, a surgical aperture 136 extending through the base sheet 124, and an attachment zone 138 located on the lower surface 132. The fifth embodiment of the drape 120 differs from the first embodiment of the drape 40, in that the fifth embodiment 120 has a guide 140 extending across (i.e., generally confronting or intersecting at an angle) the surgical aperture 136 and is located on the upper surface 128 of the base sheet 124. The inventor has found that providing a guide 140 on the upper surface 128 of the base sheet 124 may assist the physician with placement of the base sheet 124 on the eye or operative area of the patient. Proper placement of the base sheet 124 is especially advantageous to minimize the anxiety of claustrophobic patients when the vision of one or both eyes becomes obscured by the base sheet 124 (which may occur when the sheet 124 is not properly aligned with the eyes of the patient).

In FIG. 9, it can be seen that the guide 140 extends across a center or central region of the surgical aperture 136 substantially perpendicular to a length of the elongate, surgical aperture 136. It will be appreciated that there may be multiple guides 140 extending across the surgical aperture 136, and such guides 140 may be positioned at different angles with respect to the surgical aperture 136 than shown in FIG. 9. The guide 140 illustrated in FIG. 9 has the form of a strap that is attached to the upper surface 128 of the base sheet 124. The strap may be removed, cut, or severed by the physician after the attachment of the base sheet 124 to the patient. FIG. 10 illustrates a variation of the fifth embodiment wherein the guide 140 has the form of an indicium applied to the upper surface 128 of the base sheet. The indicium is a permanent ink that is applied across the surgical aperture 136 on the upper surface 128. The drape 120 having such an indicium may be more easily manufactured than that having a strap, and further prevents against potential flexing of the strap, which may impede accurate location of the base sheet 124 on the patient.

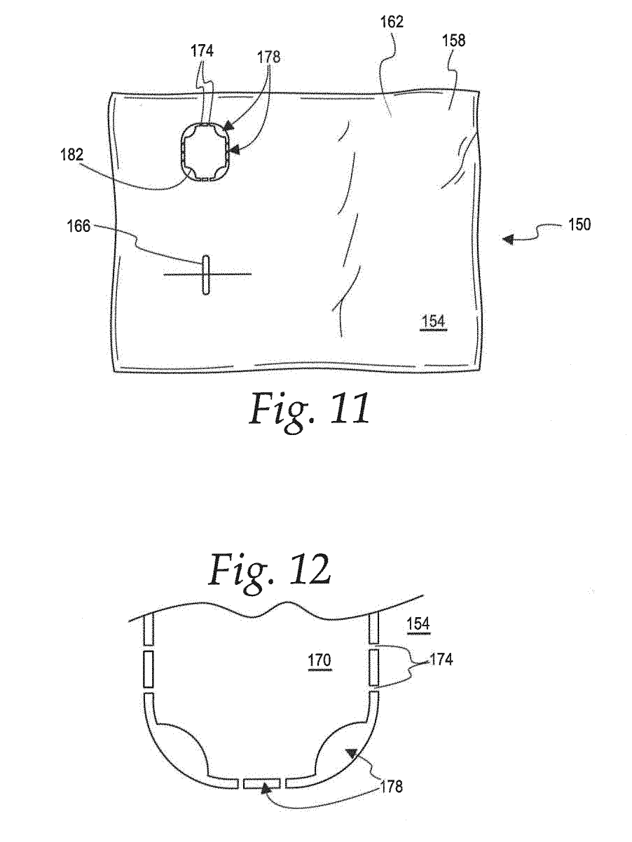

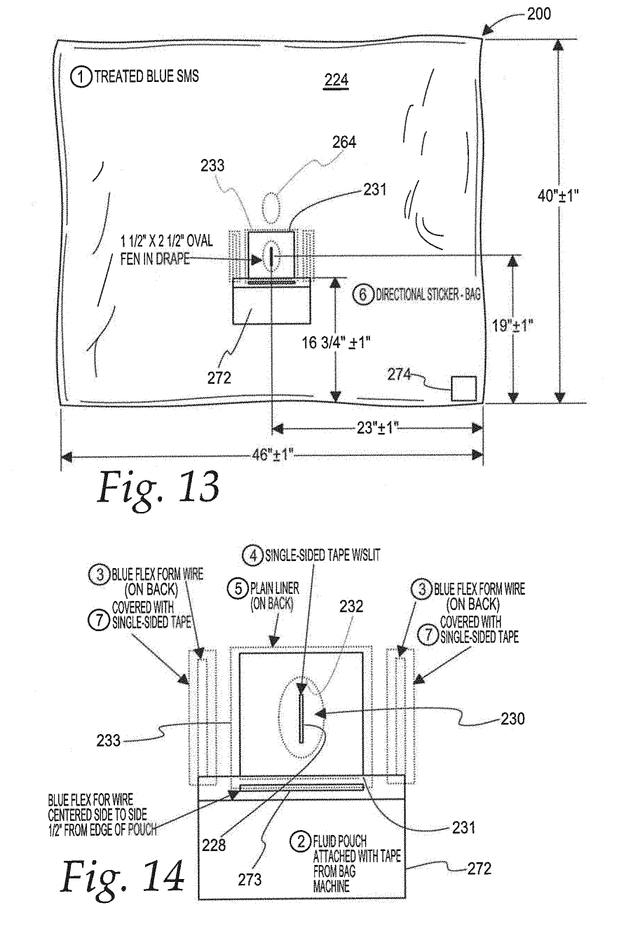

FIGS. 11 and 12 illustrate a sixth embodiment of an ophthalmic drape 150 according to the present invention. The sixth embodiment of the drape 150 functions in the same manner as the first embodiment of the drape 40 discussed above, and the drape 150 has a base sheet 154 with an upper surface 158, a lower surface 162, a surgical aperture 166 extending through the base sheet 154, and an attachment zone 158 located on the lower surface 162. The sixth embodiment of the drape 150 differs from the first embodiment of the drape 40, in that the sixth embodiment 150 has a removable flap 170 that is connected to the base sheet 154 by a plurality of frangible bridges 174. Non-surgical slots or apertures 178 are located between neighboring bridges 174, and it will be understood that the non-surgical apertures 178 serve to define the bridges 174.

The removable flap 170 is generally polygonal in shape, and is sized such that the patient cannot see the base sheet 154 when the flap 170 is removed from a remainder of the base sheet 154. The flap 170 may be further provided with one or more concave regions or recesses 182 (FIG. 11) to allow the physician to more easily grasp and remove the flap 170.

The inventor has found that providing a removable flap 170 together with the base sheet 154 may assist the physician to minimize the anxiety of claustrophobic patients when the vision of one or both eyes becomes obscured by the base sheet 154. The removable flap 170 is configured to be easily torn away from the remainder of the base sheet 154 by severance of the bridges 174 to expose the eye of the patient that is not undergoing a surgical procedure. Provision of the flap 170 allows for a single drape 150 to be utilized for both patients who may not experience claustrophobia and those patients who do experience claustrophobia.

While the flap 170 is preferably formed from the same flexible material as the base sheet 154, it will be understood that the flap 170 may be separately attached or fixed to the base sheet 154, such as by adhesive, as a tape or separable laminate, or hingedly connected via heat welding, etc. Furthermore, while the flap 170 is connected to a remainder of the base sheet 154 by eight frangible bridges 174 defined by nine non-surgical slots 178, it will be appreciated that a greater or fewer number of bridges 174 or slots 178 may be provided depending on the material of the base sheet 154 and size and strength of the individual bridges 174.

In another aspect of the present invention, a folded ophthalmic drape is provided to facilitate initial positioning of the drape on a patient, and subsequent spreading of the drape of over the patient.

In accordance with this aspect of the invention, the folded ophthalmic surgical drape, designated 200, comprises a rectangular base sheet 224 of thin, flexible material, wherein the base sheet includes an upper surface, and a lower surface for contacting the patient. The rectangular sheet includes a least one edge, typically opposite upper and lower edges, and opposite side edges. As noted, the base sheet may preferably comprise a composite non-woven SMS fabric.

The drape 200 includes a surgical aperture 228 formed through said base sheet, with the surgical aperture being arranged to be located proximal a first eye of the patient. In this embodiment, the surgical aperture 228 has a slit-like configuration and is generally elongated, and is defined by a minimally-adhesive film layer 231 affixed to the upper surface of the base sheet 224. The film layer 231 extends beyond the margins of an opening 232 defined by the base layer, and is adhered to the upper surface of the base layer at the region surrounding the opening 232. The lower surface of the film layer, having the minimally-adhesive coating thereon, is thus exposed through the opening 232 in the base layer 224 to provide the attachment zone 230 of the base layer.

The attachment zone 230 is thus provided, by the lower surface of the adhesive film 231, on the lower surface of the base sheet, proximal the surgical aperture. The attachment 230 zone provided by the adhesive film 231 at least partially surrounds the surgical aperture 228. Because the lower surface of the film layer 231 is exposed through the opening 232, a release layer 233, positioned on the lower surface of the base sheet 224, is removably adhered to the adhesive film 231 at the opening 232 in the base layer. The release line serves to maintain the freshness and efficacy of the adhesive, and serves to cover the slit-like surgical aperture 228.

The surgical aperture 228 of the drape 200 is positioned intermediate of a first pair of opposite, marginal portions 282 of the drape, and intermediate a second pair of opposite, marginal portions 284 of the drape, with the surgical aperture thus positioned generally centrally of the drape.

In accordance with this aspect of the invention, each of the marginal portions of the drape includes at least one fold to thereby facilitate placement of the folded drape on a patient with the surgical aperture positioned at the first eye of the patient. Thereafter, the first and second pairs of marginal portions can be unfolded to spread said drape to cover the patient.

In the preferred embodiment, each of the marginal portions 282, 284 comprises a plurality of folds, with it being particularly preferred that each plurality of folds comprises an accordion-fold, wherein the drape material is folded back and forth on itself to form a plurality of folded pleats. Notably, the accordion-folds in the first pair of marginal portions 282 are formed prior to the accordion-folds in the second pair of marginal portions 284, so that the accordion-folds in the second pair of marginal portions overlap, and further fold, the accordion-folds in the first pair marginal portions. It is particularly preferred that the first pair of marginal portions 282 be respectively positioned above and below the surgical aperture 228 in the drape, with the second pair of marginal portions 284 being unfolded first to spread the drape laterally over the patient, laterally of the first eye, or other surgical site, and the first pair of marginal portions 282 thereafter being unfolded to spread the drape over the patient above and below the first eye.

In another aspect of the present invention, a non-surgical aperture can be formed through the base sheet 224, with the non-surgical aperture being positionable proximal a second eye of the patient. In this embodiment, a removable flap 264 is provided in the base sheet 224, and can selectively opened to provide a non-surgical aperture, as desired. As noted, the provision of second aperture can desirably act to minimize claustrophobic sensations experienced by the patient.

As in a previous embodiment, this embodiment of the present surgical drape includes a pocket-like pouch or compartment 272 positioned adjacent to the surgical aperture 228 for use by the physician as desired. As illustrated, the compartment 272 can be provided with a deformable polymer-coated wire member 273, which can be deformed and shaped to hold the mouth of the compartment in a generally opened condition. It will be noted that efficient positioning of the drape 200, with the desired positioning of the compartment 272, is facilitated by the provision of indicia 274 on the upper surface of the base layer 224. As will be further described, the indicia indicating the position of the compartment 272 is readily visible in the folded configuration of the drape, thus assisting surgical personnel in positioning and deployment of the drape.

This embodiment also illustrates the optional provision of a pair of further deformable wire members 275, 275, respectively positioned above and below the surgical aperture 228. These wire members 275, 275, which can be secured to the upper surface of the base sheet 224, can be readily deformed to shape and contour the drape in the region of adhesive film 231 to fit the drape to the patient.

It is within the purview of the present invention to provide an additional minimally adhesive film such as 231 in the region of the drape to be positioned proximal to the second, non-surgical eye of the patient. In this embodiment, the second minimally adhesive film can be covered with a removable flap like flap 264, with both of the adhesive films adhesively applied to the patient, and the drape subsequently unfolded. If desired, a pair of the comparts 272 can be provided for respective positioning at both eyes of the patient.

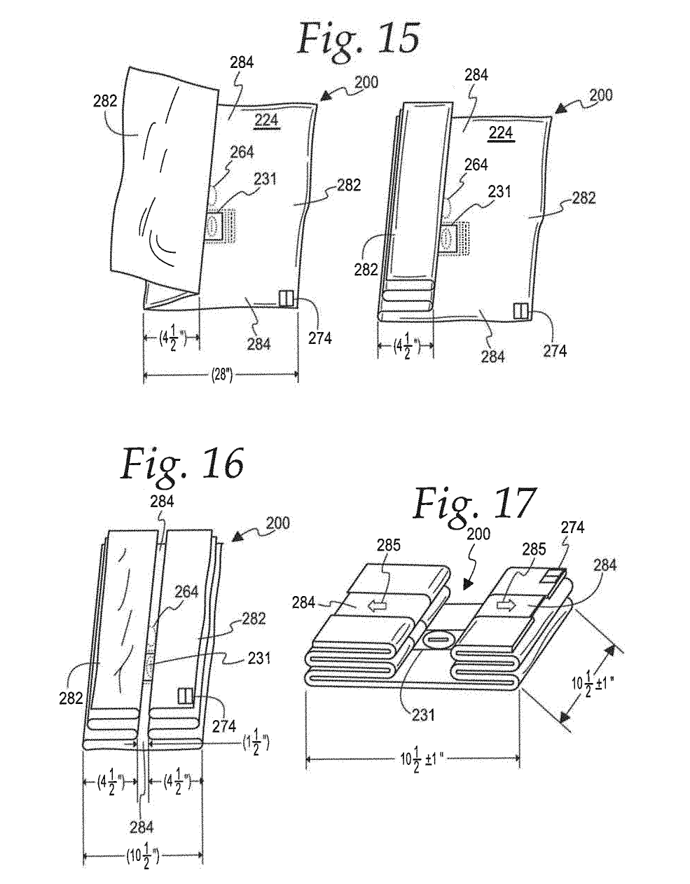

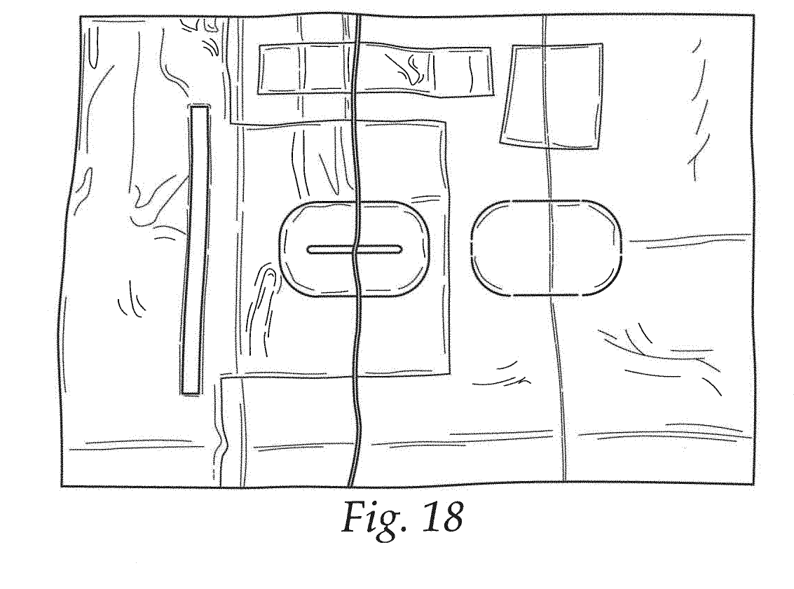

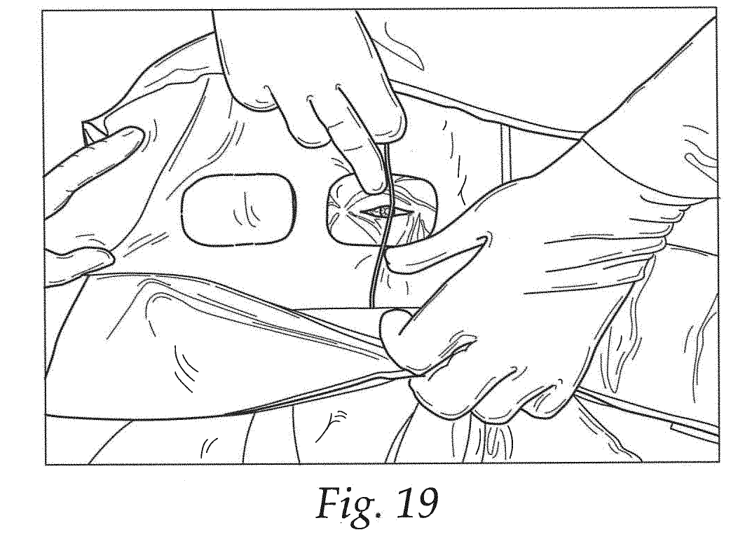

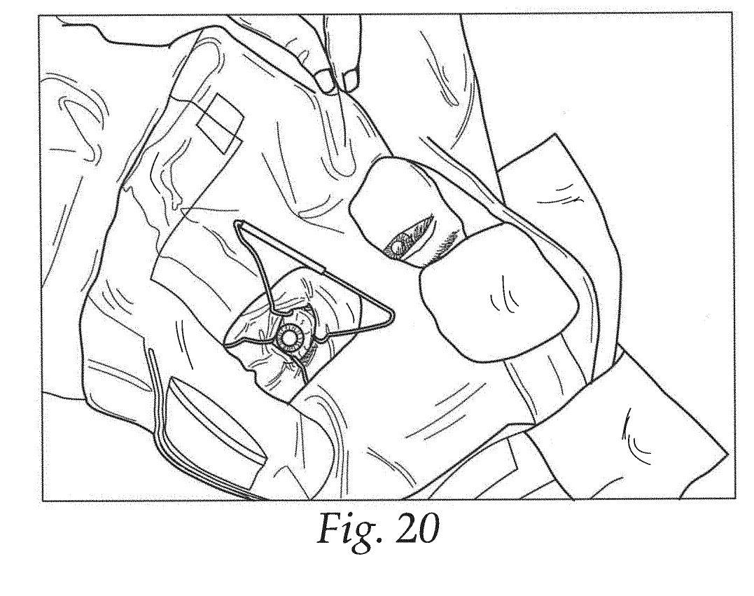

A method of folding the ophthalmic surgical drape 200 in accordance with the present invention will now be described. As noted, the folded surgical drape 200 facilitates efficient and accurate initial positioning of the drape on a patient, and permits the drape to be efficiently draped and spread to cover the patient. The sequence of folding steps is diagrammatically illustrated in FIG. 15-17.

The present method comprises the steps of providing the rectangular base sheet 224 of thin, flexible material, with the base sheet having an upper surface, a lower surface for contacting a patient, and at least one edge. The base sheet can be provided with the surgical aperture 228 formed through the base sheet 224, with surgical aperture 228 being positioned to be located proximal a first eye of the patient. The surgical aperture is positioned intermediate of the first pair of opposite, marginal portions 282 of the drape, and intermediate of the second pair of opposite, marginal portions 284 of said drape.

The drape is provided with an attachment zone on the lower surface of the base sheet 224, with the attachment zone proximal the surgical aperture 228. The attachment zone at least partially surrounds the surgical aperture, with the attachment zone configured for attaching the base sheet lower surface to the patient. As noted, the attachment zone is provided by the lower surface of film layer 231 which is exposed through opening 232 in the base layer 224.

The present method contemplates forming at least one fold in each of the marginal portions 282, 284 of the drape 200, to thereby form a folded drape, thereby facilitating placement of the folded drape 200 on a patient with the surgical aperture 228 positioned at the first eye of the patient. The first and second pairs of marginal portions can thereafter be unfolded to spread the drape to cover the patient. The method forming of folding the ophthalmic drape preferably includes forming each of the marginal portions 282, 284 with a plurality of folds, and more preferably includes forming each of the plurality of folds in each of said marginal portions as accordion-folds. This is preferably effected such that the accordion-folds are formed in the first pair of marginal portions 282 prior to forming accordion-folds in the second pair of marginal portions 282, so that the accordion-folds in the second pair of marginal portions 282 overlap and include the accordion-folds in the first pair marginal portions 282.

FIG. 15 illustrates the initiation of the folding steps, wherein one of the first marginal portions 282 is accordion-folded to form a plurality of pleats in the surgical drape. FIG. 16 illustrates the drape 200 in a partially folded condition, in which accordion-folds have been formed in each of the first marginal portions 282, prior to effecting folding of the second marginal portions 284.

FIG. 17 shows the completion of the accordion-folding of the second marginal portions 284, such that these accordion-folds overlap and include the previously folded first marginal portions 282 (note orientation of indicia 274.)

The method of forming the ophthalmic drape in accordance with this aspect of the present invention is preferably effected such that the first pair of marginal portions 282 are respectively positioned above and below the surgical aperture defined by adhesive film 231. By this preferred orientation, the second pair of marginal portions 282 can be unfolded first, from the folded configuration of the drape shown in FIG. 17, to spread the drape over the patient laterally of the first eye, or other surgical site. Indicia 285 on the second pair of lateral margins 284 indicate this initial unfolding step. The first pair of marginal portions 282 can thereafter being unfolded to spread the drape over the patient above and below the first eye.

After the drape 200 has been placed on the patient, the drape can be positioned and secured to the patient with the attachment zone provided by the minimally-adhesive film 231. The minimally-adhesive nature of the film 231 promotes the comfort of the patient during removal to the film.

For securement, the patient is instructed to open their eyes widely, and surgical personnel hold the drape 200 by stretching the film 231. The middle finger of the hand holding the drape is positioned at the center of the slit-like surgical aperture 228, and the surgical aperture is positioned parallel to the eye lids of the patient.

The procedure continues by bringing the upper edge of the surgical aperture 228 to the lower edge of the cornea of the patient's eye. The film 231 is moved closer to the patient's eye by pressing the film generally above the surgical aperture, generally at the cornea of the patient's eye.

The film 231 is next attached to the upper eyelid of the patient, and the upper eyelid of the patient retracted upwardly. The lower edge of the surgical aperture is then place at the upper edge of the cornea of the patient's eye. The film is then placed closer to the patient's eye by pressing generally at the patient's cornea, and the film is attached to the lower eyelid and retracted downwardly.

Sufficient margin of the film 231 is required to cover the eyelid edge completely. The width of the margin should be constant. The film 231 should be pressed and held at the regions on either side of the patient's eye, and the drape 200 then opened by unfolding. The speculum is placed to cover the lid margin by the film 231. The upper and lower lids are completely covered by the film. By blocking the Meibomian secretion, a wide and clean surgical field is provided.

For those patients who prefer that their whole face not be covered, a drape including a non-surgical aperture 264 can be used. The drape can be placed in the same manner described above, and the flap provided at non-surgical aperture 264 easily removed.

It is contemplated that any of the above-described embodiments may include a nose clip, or like spacing element, in order to provide h eight and clearance between the drape and the patient to facilitate convenient and comfortable breathing by the patient.

In the foregoing description, the present surgical drape has been described in connection with ophthalmic surgical procedures, but it is to be appreciated that the present surgical drape, including an attachment zone provided by a minimally-adhesive film, can be advantageously employed for other types of surgical procedures.

By way example, central venous catheterization (CVC) requires maximal sterile barrier precautions to prevent catheter-related bloodstream infection, which is associated with preventable morbidity and mortality. Disposable surgical drapes are recommended as a standard of care; however, those specifically meant for this purpose are neither freely available nor cost-effective. The present disposable drape can be advantageously employed for this purpose.

The present disposable surgical drape can be provided with an average size of 70 cm×70 cm, with an adhesive transparent area, provided by adhesive film 231, of 7 cm×9 cm. Compartment 272, such as comprising a plastic bag, is provided attached to the lateral border of the adhesive area. It covers patient's head and the thorax, and the rest of the body can be covered with a plain drape. The transparent adhesive area 231 is adequate to expose all anatomical landmarks and provides good-quality images when using ultrasound guidance, now considered a current standard of care for internal jugular vein cannulation. A small area may be cut out in the adhesive surface 231 as per the requirement to provide a surgical aperture. This is best done before opening up the surgical drape by removing the release layer 233 covering the adhesive film 231 and making a desired cut using a stab knife blade. The adhesive area is placed on the procedure site such that the attached plastic bag is placed on the lateral aspect of the neck and the drape is then opened to cover the head and the thorax.

The present disposable surgical drape provides an ideal option and ensures an optimum level of sterility. The adhesive area provided by adhesive film 231 acts to keep the drape in stable position, thereby avoiding the requirement of multiple drapes. Most of the disposable drapes are made of low-lint and abrasion-resistant fabric and have level 4 liquid barrier performance because of their non-perforated design and hence have poor absorbent quality as compared to the linen cloth drapes. The polyethylene drapes used in the absence of disposable drapes are completely non-absorbent and do not remain stable in place. The plastic bag which provides compartment 272 adjacent to the adhesive area of the surgical drape is designed to collect the irrigation fluid; during internal jugular vein annulation, it collects the trickle of blood that occurs after dilatation of the subcutaneous tract, preventing soiling and contamination of the neck and the shoulder area and the trickle over the drape down to the floor on the operator's feet.

The disposable surgical drape is more cost-effective as opposed to the one dedicated for CVC.

The present surgical drape can also be used successfully for subclavian, femoral and peripherally inserted central catheter line catheterizations. It can also be used for isolation during spinal or epidural anesthesia procedures and other regional blocks. During neuraxial blockade, the length of the transparent adhesive area should be placed along the length of the spine so that in case of difficulty one can easily have access to 2-3 interspinous spaces. The plastic bag of compartment 272 should be placed in the caudal direction. The best benefit is seen in an epidural procedure as on removing the Touhy needle after the insertion of the catheter; it is common to encounter a trickle of blood down the back soiling the operating table.

Use of the present surgical drape is also recommended for routine use for various procedures in anesthesia and the intensive care unit.

The illustrated preferred embodiments are included herein for descriptive purposes only and are not to be interpreted as limiting in any way of the broadest concepts of the present invention.

1. A disposable surgical drape comprising:
a base sheet of flexible material, said base sheet having an upper surface, a lower surface for contacting a patient;
a surgical aperture formed through said base sheet, said surgical aperture having a length, and said surgical aperture for being located proximal to a first eye of the patient;
an attachment zone formed on said base sheet lower surface, said attachment zone being located proximal to said surgical aperture, said attachment zone at least partially surrounding said surgical aperture, said attachment zone for attaching said base sheet lower surface to the patient to fix at least a portion of said base sheet with respect to the patient; and
a guide extending across said surgical aperture, said guide being located on said upper surface of said base sheet.

2. The disposable surgical drape of claim 1 wherein said guide has the form of a strap attached to said base sheet upper surface.

3. The disposable surgical drape of claim 1 wherein said guide has the form of at least one indicium applied to said base sheet upper surface.

4. The disposable surgical drape of claim 1 wherein said a base sheet further comprises a compartment on said upper surface.

5. The disposable surgical drape of claim 1 wherein said base sheet further comprises a removable flap for being located proximal a second eye of the patient, said removable flap being configured to provide an unobstructed view to the second eye of the patient when said removable flap is removed from said base sheet.

6. The disposable surgical drape of claim 1 wherein said attachment zone is an adhesive.

7. The disposable surgical drape of claim 1 wherein said attachment zone fully surrounds said surgical aperture.

8. A disposable surgical drape, comprising:
a base sheet of flexible material, said base sheet having an upper surface, a lower surface for contacting a patient;
a surgical aperture formed through said base sheet, said surgical aperture for being located proximal to a first eye of the patient;
an attachment zone formed on said base sheet lower surface, said attachment zone being located proximal to said surgical aperture, said attachment zone at least partially surrounding said surgical aperture, said attachment zone for attaching said base sheet lower surface to the patient; and
a removable flap spaced from said surgical aperture and for being located proximal a second eye of the patient.

9. The disposable surgical drape of claim 8, wherein said removable flap is connected to said base sheet by at least one frangible bridge defined by a plurality of non-surgical apertures in said base sheet, whereby removal of said removable flap from said base sheet exposes the second eye of the patient.

10. The disposable surgical drape of claim 8, further comprising a guide extending across said surgical aperture, said guide being located on said upper surface of said base sheet.

11. The disposable surgical drape of claim 8 wherein said a base sheet further comprises a compartment on said upper surface.

12. The disposable surgical drape of claim 8, wherein said attachment zone is an adhesive.

13. A folded disposable surgical drape, comprising:
a rectangular base sheet of thin, flexible material, said base sheet having an upper surface, a lower surface for contacting a patient, and at least one edge; and
an attachment zone on said base sheet lower surface, said attachment zone being configured for attaching said base sheet lower surface to a surgical site of the patient;
said attachment zone being positioned intermediate of a first pair of marginal portions of said drape, and a second pair of marginal portions of said drape, wherein each of said marginal portions includes at least one fold, to facilitate placement of said folded drape on a patient with said attachment zone affixed to the surgical site of the patient so that said first and second pairs of marginal portions can be unfolded to spread said drape to cover the patient.

14. A folded disposable surgical drape in accordance with claim 13, wherein said drape defines an opening, and includes an adhesive film layer adhered to said upper surface of said base sheet so that said film layer is exposed through said opening in said base layer to provide said attachment zone.

15. A folded surgical drape in accordance with claim 14, including a surgical aperture formed through adhesive film layer, said surgical aperture being positionable proximal the surgical site of the patient.

16. A folded disposable surgical drape in accordance with claim 13, wherein each of said marginal portions comprises a plurality of folds.

17. A folded disposable surgical drape in accordance with claim 16, wherein said plurality of folds in each of said marginal portions comprise accordion-folds.

18. A folded disposable surgical drape in accordance with claim 17, wherein said accordion-folds in said first pair of marginal portions are formed prior to said accordion-folds in said second pair of marginal portions, so that said accordion-folds in said second pair of marginal portions overlap said accordion-folds in said first pair marginal portions.

19. A folded disposable surgical drape in accordance with claim 18, wherein said first pair of marginal portions are respectively positioned above and below said attachment zone, said second pair of marginal portions being unfolded first to spread said drape laterally over the patient, said first pair of marginal portions thereafter being unfolded to spread said drape over said upwardly and downwardly of the surgical site of the patient.

20. A folded disposable surgical drape in accordance with claim 13, including a compartment positioned on the upper surface of said base layer proximal said surgical aperture.

21. A folded disposable surgical drape in accordance with claim 13, including a removable flap to provide a non-surgical aperture formed through said base sheet, said non-surgical aperture for being located proximal a second eye of the patient.

22. A method of folding a disposable surgical drape, comprising the steps of:
providing a rectangular base sheet of thin, flexible material, said base sheet having an upper surface, a lower surface for contacting a patient, and at least one edge;
providing a surgical aperture formed through said base sheet, said surgical aperture being positionable proximally of a first eye of the patient, including positioning said surgical aperture intermediate of a first pair of marginal portions of said drape, and intermediate of a second pair of marginal portions of said drape;
providing an attachment zone on said base sheet lower surface, said attachment zone being proximal said surgical aperture, said attachment zone at least partially surrounding said surgical aperture, said attachment zone being configured for attaching said base sheet lower surface to the patient; and
forming at least one fold in each of said marginal portions of said drape, to form a folded drape to facilitate placement of said folded drape on, a patient with said surgical aperture positioned at said first eye of said patient, so that said first and second pairs of marginal portions can be unfolded to spread said drape to cover the patient.

23. A method forming a folded disposable surgical drape in accordance with claim 22, including forming each of said marginal portions with a plurality of folds, including forming each of said plurality of folds in each of said marginal portions as accordion-folds, including forming said accordion-folds in said first pair of marginal portions prior to forming said accordion-folds in said second pair of marginal portions, so that said accordion-folds in said second pair of marginal portions overlap and include said accordion-folds in said first pair marginal portions.

24. A method of forming a folded disposable surgical drape in accordance with claim 22, including respectively positioning said first pair of marginal portions above and below said surgical aperture, so that said second pair of marginal portions can be unfolded first to spread said drape over the patient laterally of said first eye, with said first pair of marginal portions thereafter being unfolded to spread said drape over said patient above and below said first eye.
